SEQUENCE LISTING (12 sequences)

SEQ ID NO: 1 - protein, 360 aa, Artificial Sequence
  OCT4
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 317 aa, Artificial Sequence
  SOX2
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 3 - protein, 454 aa, Artificial Sequence
  c-Myc
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 4 - protein, 504 aa, Artificial Sequence
  KLF4
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 5 - DNA, 17 bp, Artificial Sequence
  OCT-4_F
tctcgccccctccaggt

SEQ ID NO: 6 - DNA, 17 bp, Artificial Sequence
  OCT-4_R
ctgcttcgccctcaggc

SEQ ID NO: 7 - DNA, 20 bp, Artificial Sequence
  Nanog_F
aaagaatcttcacctatgcc

SEQ ID NO: 8 - DNA, 20 bp, Artificial Sequence
  Nanog_R
gaaggaagaggagagacagt

SEQ ID NO: 9 - DNA, 24 bp, Artificial Sequence
  REX-1_F
cagatcctaaacagctcgcagaat

SEQ ID NO: 10 - DNA, 23 bp, Artificial Sequence
  REX-1_R
gcgtacgcaaattaaagtccaga

SEQ ID NO: 11 - DNA, 20 bp, Artificial Sequence
  GAPDH_F
gagtccactggcgtcttcac

SEQ ID NO: 12 - DNA, 20 bp, Artificial Sequence
  GAPDH_R
ttcacacccatgacgaacat

(19) United States
(12) Patent Application Publication
(10) Pub. No.: US 2021/0062144 A1
(43) Pub. Date: Mar. 4, 2021

(54) PLACENTA-DERIVED CELL-CONDITIONED MEDIUM FOR INDUCING DEDIFFERENTIATION INTO INDUCED PLURIPOTENT STEM CELLS FROM SOMATIC CELLS AND METHOD FOR INDUCING DEDIFFERENTIATION USING THE SAME

(71) Applicant: KOREA UNIVERSITY RESEARCH AND BUSINESS FOUNDATION, Seoul (KR)

(72) Inventors: Byoung Soo KIM, Seoul (KR); Seung Jin LEE, Seoul (KR); Ka Won KANG, Seoul (KR)

(21) Appl. No.: 16/956,798

(22) PCT Filed: Dec. 21, 2018
(86) PCT No.: PCT/KR2018/016519

§ 371 Date: Jun. 22, 2020

(30) Foreign Application Priority Data
Dec. 22, 2017 (KR) 10-2017-0178706

(51) Int. Cl. C12N 5/073; C12N 5/074

(52) U.S. Cl. CPC C12N 5/0605; C12N 5/0696; C12N 2501/602; C12N 2501/603; C12N 2506/1307; C12N 2502/025; C12N 2506/28; C12N 2501/604; C12N 2501/606

(57) ABSTRACT

The present disclosure relates to a placenta-derived cell-conditioned medium for inducing dedifferentiation into induced pluripotent stem cells from somatic cells and a method for inducing dedifferentiation using the same. When the placenta-derived cell-conditioned medium for inducing dedifferentiation according to the present disclosure is employed, personalized dedifferentiation stem cells can be stably established using a medium composed of human-derived products only. Provision of a human placenta-derived environment similarly represents an in vivo environment and allows the production of a cell therapy product without problems for clinical application.

### FIELD

The present invention relates to a placenta-derived cell-conditioned medium for inducing dedifferentiation into induced pluripotent stem cells from somatic cells and a method for inducing dedifferentiation using the same.

### BACKGROUND

In order to produce stem cell therapeutic agents, large scale stem cell culture in vitro being a source thereof must be essentially carried out, and they must be safe and economical to be used as cell therapeutic agents in clinical practice.

However, for the proliferation culture of human induced pluripotent stem cells used at present, the method of using animal-derived support cells and the method of culturing in a container coated with a special gel containing animal-derived products may cause safety problems due to heterologous protein contamination. In case of using an expensive special gel, it is not suitable for mass production from economic perspectives.

When a placenta-derived cell-conditioned medium is employed, induced pluripotent stem cells can be cultured using animal-free and feeder-free culture system, and proliferation and differentiation of pluripotent stem cells can be performed by the mechanism of CXCR2 which is a chemokine receptor.

Therefore, the present inventors anticipated that the placenta-derived cell-conditioned medium, which had been proven to be useful for culturing stem cells, can be utilized for the development of cell therapeutic agents by applying it to the dedifferentiation into induced pluripotent stem cells.

### SUMMARY

### Technical Problem

The present inventors have made intensive researches to develop a placenta-derived cell-conditioned medium for inducing dedifferentiation into induced pluripotent stem cells (iPS) from somatic cells.

As a result, they have found that the dedifferentiation efficiency of induced pluripotent stem cells from somatic cells can be increased as compared with the case where a placenta-derived cell-conditioned medium is not used, thereby completing the present disclosure.

Therefore, it is one object of the present disclosure to provide a placenta-derived cell-conditioned medium for inducing dedifferentiation into induced pluripotent stem cells from somatic cells.

It is another object of the present disclosure to provide a method for preparing a placenta-derived cell-conditioned medium for inducing dedifferentiation into induced pluripotent stem cells from somatic cells.

It is yet another object of the present disclosure to provide a method for inducing dedifferentiation into induced pluripotent stem cells from somatic cells using the placenta-derived cell-conditioned medium for inducing dedifferentiation.

### Technical Solution

The present inventors have made intensive researches to develop a placenta-derived cell-conditioned medium for inducing dedifferentiation into induced pluripotent stem cells from somatic cells. As a result, they have found that the dedifferentiation efficiency of induced pluripotent stem cells from somatic cells can be increased as compared with the case where a placenta-derived cell-conditioned media is not employed.

Hereinafter, embodiments of the present disclosure will be described in more detail.

One aspect of the present disclosure provides a placenta-derived cell-conditioned medium for inducing dedifferentiation into induced pluripotent stem cells (iPS) from somatic cells.

The somatic cell may be transformed with a nucleic acid sequence encoding at least one protein selected from the group consisting of OCT4, SOX2, c-Myc and KLF4, and for example, it may be transformed with a nucleic acid sequence encoding the OCT4 protein, a nucleic acid sequence encoding the SOX2 protein, a nucleic acid sequence encoding the c-Myc protein, and a nucleic acid sequence encoding the KLF4 protein.

The OCT4 protein may include an amino acid sequence of SEQ ID NO: 1, and for example, may be composed of the amino acid sequence of SEQ ID NO: 1, and may be interpreted to include sequences which have substantial identity thereto.

In addition, the nucleic acid encoding the OCT4 protein may encode the OCT4 protein including the amino acid sequence of SEQ ID NO: 1, and for example, may encode the OCT4 protein composed of the amino acid sequence of SEQ ID NO: 1, and may be interpreted to include sequences which have substantial identity thereto.

The SOX2 protein may include an amino acid sequence of SEQ ID NO: 2, and for example, may be composed of the amino acid sequence of SEQ ID NO: 2, and may be interpreted to include sequences which have substantial identity thereto.

Moreover, the nucleic acid encoding the SOX2 protein may encode the SOX2 protein including the amino acid sequence of SEQ ID NO: 2, and for example, may encode the SOX2 protein composed of the amino acid sequence of SEQ ID NO: 2, and may be interpreted to include sequences which have substantial identity thereto.

The c-Myc protein may include an amino acid sequence of SEQ ID NO: 3, and for example, may be composed of the amino acid sequence of SEQ ID NO: 3, and may be interpreted to include sequences which have substantial identity thereto.

Further, the nucleic acid encoding the c-Myc protein may encode the c-Myc protein including the amino acid sequence of SEQ ID NO: 3, and for example, may encode the c-Myc protein composed of the amino acid sequence of SEQ ID NO: 3, and may be interpreted to include sequences which have substantial identity thereto.

The KLF4 protein may include an amino acid sequence of SEQ ID NO: 4, and for example, may be composed of the amino acid sequence of SEQ ID NO: 4, and may be interpreted to include sequences interpreted to include sequences which have substantial identity thereto.

Further, the nucleic acid encoding the KLF4 protein may encode the KLF4 protein including the amino acid sequence of SEQ ID NO: 4, and for example, it may encode the KLF4 protein composed of the amino acid sequence of SEQ ID NO: 4, and may be interpreted to include sequences which have substantial identity thereto.

The substantial identity may be a sequence showing at least 60% homology, at least 70% homology, at least 80% homology, or at least 90% homology after fully aligning the target sequence with any other sequences and analyzing the aligned sequences using an algorithm commonly used in the art.

Alignment methods for comparison of sequences are known in the art, and for example, sequence analysis programs such as blastp, blastx, tblastn and tblastx can be used on the Internet using the Basic Local Alignment Search Tool (BLAST)of NCBI.

TABLE 1

| SEQ | | | |
|---|---|---|---|
| ID NO: | Name | Sequence | Note |
| 1 | OCT4 | MAGHLASDFAFSPPPGGGGDGPGGPEPGWVDPR | |
| | | TWLSFQGPPGGPGIGPGVGPGSEVWGIPPCPPPY | |
| | | EFCGGMAYCGPQVGVGLVPQGGLETSQPEGEAG | |
| | | VGVESNSDGASPEPCTVTPGAVKLEKEKLEQNPE | |
| | | ESQDIKALQKELEQFAKLLKQKRITLGYTQADVGLT | |
| | | LGVLFGKVFSQTTICRFEALQLSFKNMCKLRPLLQ | |
| | | KWVEEADNNENLQEICKAETLVQARKRKRTSIENR | |
| | | VRGNLENLFLQCPKPTLQQISHIAQQLGLEKDVVR | |
| | | VWFCNRRQKGKRSSSDYAQREDFEAAGSPFSGG | |
| | | PVSFPLAPGPHFGTPGYGSPHFTALYSSVPFPEGE | |
| | | AFPPVSVTTLGSPMHSN | |
| 2 | SOX2 | MYNMMETELKPPGPQQTSGGGGGNSTAAAAGGN | |
| | | QKNSPDRVKRPMNAFMVWSRGQRRKMAQENPK | |
| | | MHNSEISKRLGAEWKLLSETEKRPFIDEAKRLRAL | |
| | | HMKEHPDYKYRPRRKTKTLMKKDKYTLPGGLLAP | |
| | | GGNSMASGVGVGAGLGAGVNQRMDSYAHMNGW | |
| | | SNGSYSMMQDQLGYPQHPGLNAHGAAQMQPMH | |
| | | RYDVSALQYNSMTSSQTYMNGSPTYSMSYSQQG | |
| | | TPGMALGSMGSVVKSEASSSPPVVTSSSHSRAPC | |
| | | QAGDLRDMISMYLPGAEVPEPAAPSRLHMSQHYQ | |
| | | SGPVPGTAINGTLPLSHM | |
| 3 | c-Myc | MDFFRVVENQQPPATMPLNVSFTNRNYDLDYDSV | |
| | | QPYFYCDEEENFYQQQQQSELQPPAPSEDIWKKF | |
| | | ELLPTPPLSPSRRSGLCSPSYVAVTPFSLRGDNDG | |
| | | GGGSFSTADQLEMVTELLGGDMVNQSFICDPDDE | |
| | | TFIKNIIIQDCMWSGFSAAAKLVSEKLASYQAARKD | |
| | | SGSPNPARGHSVCSTSSLYLQDLSAAASECIDPSV | |
| | | VFPYPLNDSSSPKSCASQDSSAFSPSSDSLLSSTE | |
| | | SSPQGSPEPLVLHEETPPTTSSDSEEEQEDEEEID | |
| | | VVSVEKRQAPGKRSESGSPSAGGHSKPPHSPLVL | |
| | | KRCHVSTHQHNYAAPPSTRKDYPAAKRVKLDSVR | |
| | | VLRQISNNRKCTSPRSSDTEENVKRRTHNVLERQ | |
| | | RRNELKRSFFALRDQIPELENNEKAPKVVILKKATA | |
| | | YILSVQAEEQKLISEEDLLRKRREQLKHKLEQLRNS | |
| | | CA | |
| 4 | KLF4 | MAVSDALLPSFSTFASGPAGREKTLRQAGAPNNR | |
| | | WREELSHMKRLPPVLPGRPYDLAAATVATDLESG | |
| | | GAGAACGGSNLAPLPRRETEEFNDLLDLDFILSNS | |
| | | LTHPPESVAATVSSSASASSSSSPSSSGPASAPST | |
| | | CSFTYPIRAGNDPGVAPGGTGGGLLYGRESAPPP | |
| | | TAPFNLADINDVSPSGGFVAELLRPELDPVYIPPQQ | |
| | | PQPPGGGLMGKFVLKASLSAPGSEYGSPSVISVSK | |
| | | GSPDGSHPVVVAPYNGGPPRTCPKIKQEAVSSCT | |
| | | HLGAGPPLSNGHRPAAHDFPLGRQLPSRTTPTLG | |
| | | LEEVLSSRDCHPALPLPPGFHPHPGPNYPSFLPDQ | |
| | | MQPQVPPLHYQGQSRGFVARAGEPCVCWPHFGT | |
| | | HGMMLTPPSSPLELMPPGSCMPEEPKPKRGRRS | |
| | | WPRKRTATHTCDYAGCGKTYTKSSHLKAHLRTHT | |
| | | GEKPYHCDWDGCGWKFARSDELTRHYRKHTGHR | |
| | | PFQCQKCDRAFSRSDHLALHMKRHF | |

The somatic cell may be at least one selected from the group consisting of endothelial cells, epithelial cells and placental cells.

The placenta-derived cell may be a placenta-derived fibroblast-like cell, which is isolated from the human chorionic plate and cultured.

The placenta-derived cell-conditioned medium may include human placenta-derived cells cultured in a cell growth medium.

Another aspect of the present disclosure relates to a method for preparing a placenta-derived cell-conditioned medium for inducing dedifferentiation, including the following steps:

a placenta-derived cell culturing step of culturing human placenta-derived cells in a cell growth medium supplemented with a culture solution; and

a culture solution collecting step of collecting the culture solution including the human placenta-derived cell culture from the cell growth medium.

The placenta-derived cell may be a placenta-derived fibroblast-like cell, which is isolated from the human chorionic plate and cultured.

The culture solution of the culturing step may be Dulbecco's modified Eagle's medium (DMEM)/F-12, and for example, it may be DMEM containing 10% FBS, 10% penicillin and 10% sodium pyruvate, or a high-glucose medium.

The culture solution may further include a serum replacement agent, and may be, for example, KnockOut™ Serum Replacement (KnockOut™ SR), but is not limited thereto.

The culture solution collected in the collection step may include a placenta-derived cell culture, for example, a human placenta-derived cell culture.

Yet another aspect of the present disclosure relates to a method for inducing dedifferentiation into induced pluripotent stem cells (iPS) from somatic cells, including the following steps:

a somatic cell transformation step of transducing a nucleic acid sequence encoding at least one protein selected from the group consisting of OCT4, SOX2, c-Myc and KLF4 into somatic cells; and

a somatic cell culturing step of culturing the transformed somatic cells in a placenta-derived cell-conditioned medium.

The somatic cell may be at least one selected from the group consisting of endothelial cells, epithelial cells and placental cells.

The placenta-derived cell may be a placenta-derived fibroblast-like cell, which is isolated from the human chorionic plate and cultured.

The method for inducing dedifferentiation may further include a stem cell isolation step of isolating stem cells from colonies formed during the somatic cell culturing step.

The stem cell isolation step may be performed by staining with a stem cell marker, and for example, it may be performed by staining with Tra-60, alkaline phosphatase, SSEA4, TRA-1-60 and TRA-1-80, and may be isolated through a live stain in order to confirm whether the formed colonies are stem cells.

The method for inducing dedifferentiation may further include a stem cell activity verification step of confirming the activity of at least one protein selected from the group consisting of OCT-4, NANOG, SSEA-4 and Tra-81 in the stem cells isolated from colonies.

The activity verification step may be performed by comparing the reprogramming efficiency through an alkaline phosphatase staining after transducing a dedifferentiation factor for 7 days and inducing dedifferentiation stem cells for 3 days in the placenta-derived cell-conditioned medium or E8, but is not limited thereto.

The method for inducing dedifferentiation may further include a stem cell differentiation ability verification step of confirming the differentiation ability into ectoderm, mesoderm or endoderm by forming embryonic cells in vitro using the stem cells isolated from colonies.

The verification of the differentiation ability into the ectoderm may be performed with at least one antibody selected from the group consisting of TUJ1, Nestin, Otx2, SOX1 and Pax6, but is not limited thereto.

The verification of the differentiation ability into the mesoderm may be performed with at least one antibody selected from the group consisting of Desmin, Brachyury, HAND1 and Snail, but is not limited thereto.

The verification of the differentiation ability into the endoderm may be performed with at least one antibody selected from the group consisting of AFP, GATA-4, SOX17, HNF4A and FOXA2, but is not limited thereto.

### Advantageous Effects

The present disclosure relates to a placenta-derived cell-conditioned medium for inducing dedifferentiation into induced pluripotent stem cells from somatic cells and a method for inducing dedifferentiation using the same. When the placenta-derived cell-conditioned medium for inducing dedifferentiation according to the present disclosure is employed, personalized dedifferentiation stem cells can be stably established using a medium composed of human-derived products only. In addition, the dedifferentiation efficiency of induced pluripotent stem cells from somatic cells can be increased as compared with the case where a placenta-derived cell-conditioned medium is not used.

### BRIEF DESCRIPTION OF THE DRAWINGS

FIG. 1 is a schematic diagram showing a process for inducing dedifferentiation into induced pluripotent stem cells from somatic cells according to an embodiment of the present disclosure.

FIG. 2 shows images showing morphological changes in the process of dedifferentiation into induced pluripotent stem cells from somatic cells according to an embodiment of the present disclosure.

FIG. 3A is an image showing the results of staining ALP, a stem cell marker for the induced pluripotent stem cells prepared by inducing dedifferentiation according to an embodiment of the present disclosure.

FIG. 3B is a graph showing the results of comparing the dedifferentiation efficiency of induced pluripotent stem cells in the placenta-derived cell-conditioned medium relative to normal medium according to an embodiment of the present disclosure.

FIG. 4A shows images showing the results of confirming the expression of the stem cell-specific gene through an immunohistochemical method for the induced pluripotent stem cells prepared by inducing dedifferentiation according to an embodiment of the present disclosure.

FIG. 4B is a graph showing the results of confirming the expression of the stem cell-specific gene through a polymerase chain reaction for the induced pluripotent stem cells prepared by inducing dedifferentiation according to an embodiment of the present disclosure.

FIG. 5A is an image showing the results of confirming the karyotype through chromosomal analysis for the induced pluripotent stem cells prepared by inducing dedifferentiation in vascular endothelial cells according to an embodiment of the present disclosure.

FIG. 5B is an image showing the results of confirming the karyotype through chromosomal analysis for the induced pluripotent stem cells prepared by inducing dedifferentiation in placental cells according to an embodiment of the present disclosure.

FIG. 5C is an image showing the results of confirming the karyotype through chromosomal analysis for the induced pluripotent stem cells prepared by inducing dedifferentiation in fibroblasts according to an embodiment of the present disclosure.

FIG. 6A is an image showing the results of confirming the differentiation ability into ectoderm, mesoderm and endoderm by forming teratomas in vitro with the induced pluripotent stem cells prepared by inducing dedifferentiation from somatic cells according to an embodiment of the present disclosure.

FIG. 6B is an image showing the results of verifying the formation of teratomas in immune-deficient mice with the induced pluripotent stem cells prepared by inducing dedifferentiation from somatic cells according to an embodiment of the present disclosure.

FIG. 6C is an image showing the results of verifying the formation of teratomas in immune-deficient mice with the induced pluripotent stem cells prepared by inducing dedifferentiation from somatic cells according to an embodiment of the present disclosure.

### BEST MODE

Placenta-derived cell-conditioned medium for inducing dedifferentiation into induced pluripotent stem cells from somatic cells and method for inducing dedifferentiation using the same.

### DETAILED DESCRIPTION

A placenta-derived cell-conditioned medium for inducing dedifferentiation into induced pluripotent stem cells (iPS) from somatic cells.

Hereinafter, the present disclosure will be described in more detail by way of Examples. However, these Examples are merely provided to more specifically describe the present disclosure, and it is obvious to those skilled in the art that, according to the gist of the present disclosure, the scope of the present disclosure is not limited to or by these examples.

### EXAMPLE 1: DEDIFFERENTION INTO INDUCED PLURIPOTENT STEM CELLS FROM SOMATIC CELLS

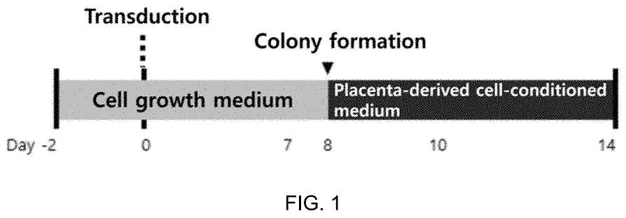

As shown in FIG. 1, endothelial cells (Primary Umbilical Vein Endothelial Cells, ATCC #PCS-100-010), epidermal Cells (Primary Dermal Fibroblasts, ATCC # PCS-201-012) and placental cells, which are three types of human somatic cells, were transformed with Sandy virus dedifferentiation factors (OCT4, SOX2, c-Myc and KLF4) and incubated in the growth medium provided for 7 days.

The placental cells were obtained from placental tissues isolated by surgical operation through a cesarean section after a written consent from a healthy pregnant woman who received therapeutic abortion at 7 weeks of pregnancy.

In detial, cells were isolated from chorionic tissues of the placenta, and the isolated placental cells were incubated in Dulbecco's modified Eagle's medium (DMEM) containing 20% fetal bovine serum (FBS), 100 U/ml penicillin and 100 g/ml streptomycin in a flask coated with 0.1% gelatin for one week.

Transformation was performed by purchasing a kit (CytoTune™-iPS 2.0 Sendai Reprogramming Kit, Life Technologies).

The transformed somatic cells were transplanted into a new culture vessel, and after 8 days, the cells were incubated in the placenta-derived cell-conditioned medium provided, and colonies were formed within 24 hours.

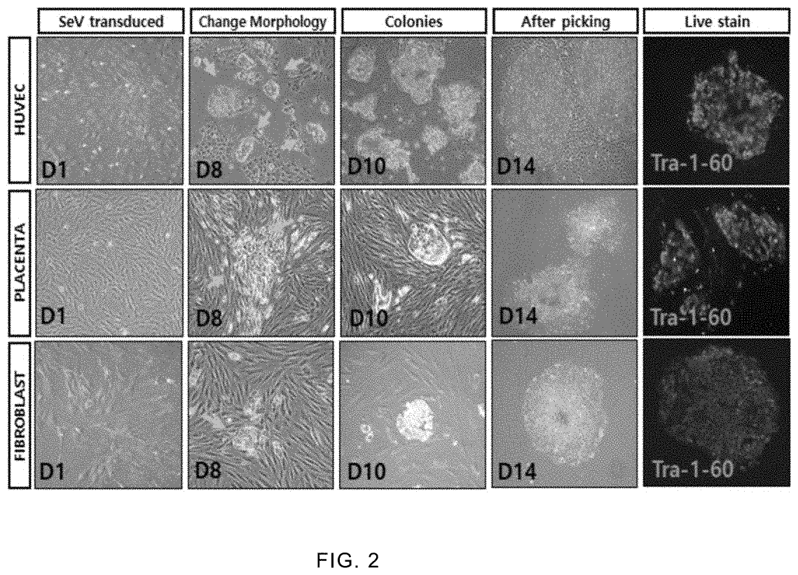

As shown in FIG. 2, among these, the dedifferentiation stem cells, which were verified by staining with Tra-60 which is a stem cell marker, were selectively isolated and cultured. At this time, a commercialized culture solution (define Essential 8 medium: E8) was used as a control.

### EXAMPLE 2: CONFIRMATION OF DEDIFFERENTIATION STEM CELLS

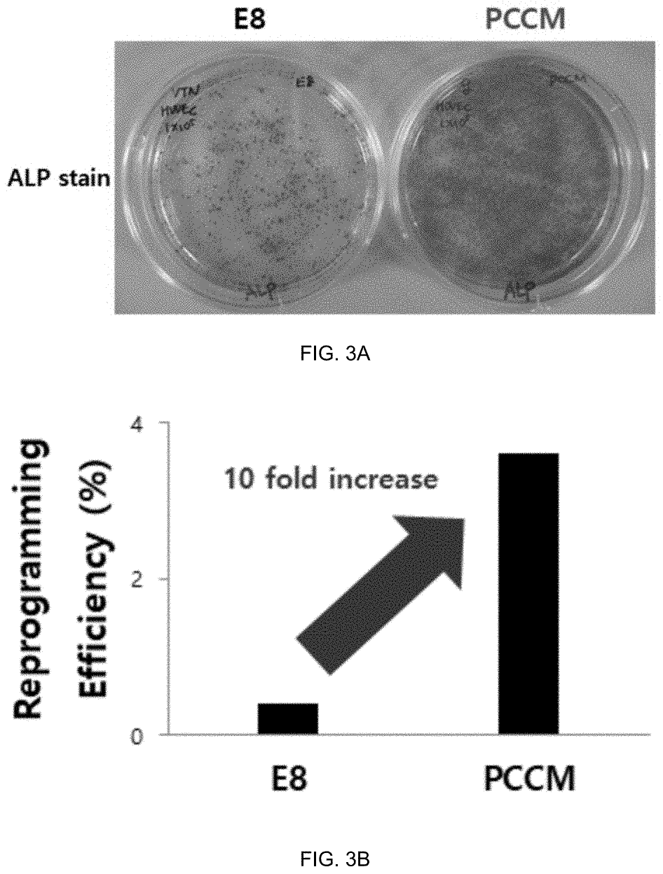

It was confirmed through an alkaline phosphatase staining whether the dedifferentiation stem cells induced from the somatic cells exhibited a self-renewal ability, which are characteristics of induced pluripotent stem cells, and the efficiency thereof was compared with a control group in which the dedifferentiation was induced in E8 medium. The alkaline phosphatase staining was performed using a kit (ES Cell Characterization kit, Chemicon International), and the results are shown in FIG. 3A. These were calculated as efficiency (%) and plotted as a graph, the results of which are shown in FIG. 3B and Table 2.

TABLE 2

| Condition | Cells plated | Colonies/well | Efficiency (%) |
|---|---|---|---|
| E8 | 1 × 10⁵ | 312 ± 0.05 | 0.312 |
| PCCM | 1 × 10⁵ | 3921 ± 0.01 | 3.921 |

As can be seen in FIG. 3A, it could also be observed with the naked eye that there were a significant number of cells showing the characteristics of the induced pluripotent stem cells in the placenta-derived cell-conditioned medium. In addition, as can be confirmed in FIG. 3B and Table 2, in a total of 100,000 cells, 312±0.05 cells showed ALP activity in E8, and 3921±0.01 cells in PCCM, and the dedifferentiation efficiency in the placenta-derived cell-conditioned medium was found to be about 10 times higher than in the control group.

### EXAMPLE 3: CONFIRMATION OF SPECIFICITY OF DEDIFFERENTIATION STEMS CELLS

### 3-1. Confirmation of Specific Marker Expression Level

The function of stem cells was verified by confirming the expression levels of OCT-4, NANOG, SSEA-4, and Tra-81, which are specific markers for induced pluripotent stem cells. In order to confirm whether the three established stem cell lines retained their properties, the expression of markers specific for dedifferentiation stem cells was confirmed using an immunofluorescence staining. First, cells were cultured on a cover slip for the immunofluorescence staining, and the expression of the stem cell-specific markers OCT-4 and SSEA-4 was measured by immunofluorescence staining.

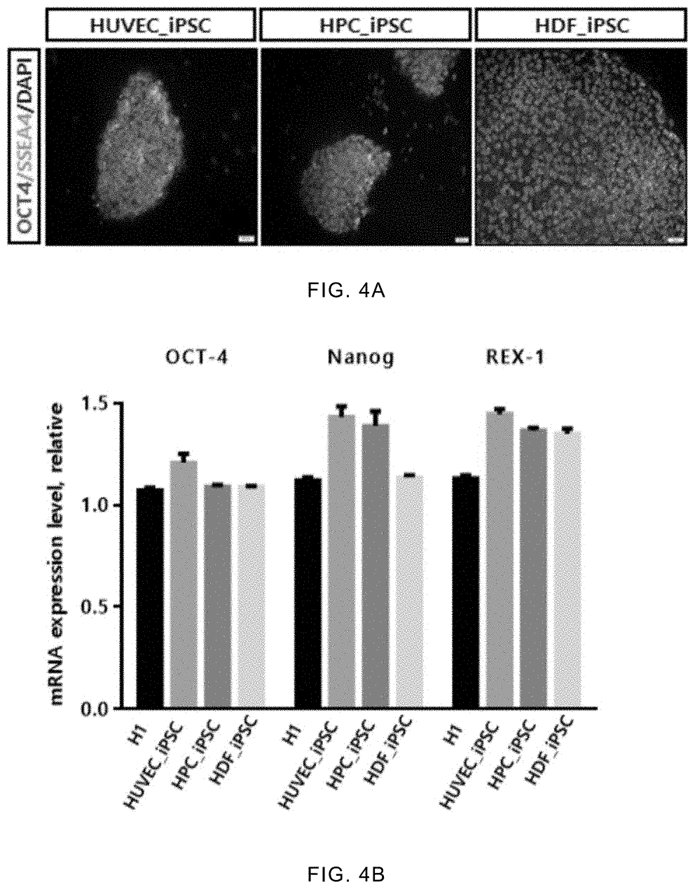

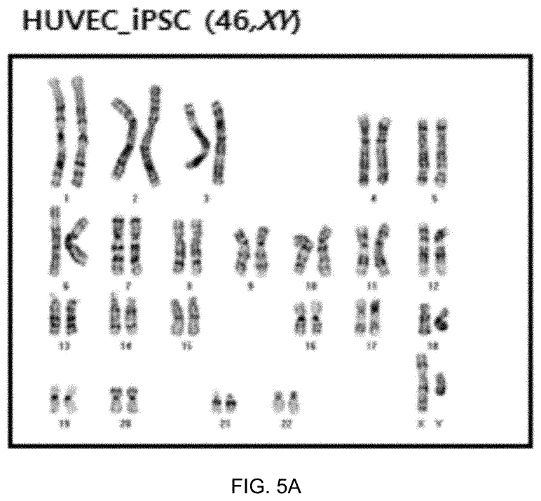

Specifically, when the cells were grown to 70 to 80%, they were fixed with 4% paraformaldehyde for 10 minutes. Then, 0.1% Triton X100 was infiltrated into the cells for 10 minutes, and the primary antibody OCT-4 (Cell Signaling Technology #2750) and SSEA-4 (Millipore # MAB4304) were diluted at 1:1000 and treated to the cells. Then, the cells were incubated overnight at 4° C. The next day, the cells were treated with the secondary antibody at room temperature for 1 hour and with 4′,6-diamidino-2-phenylindole (DAPI), allowed to stand for 5 minutes under dark conditions, and then observed under a fluorescence microscope. Then, the mRNA expression levels of OCT-4, Nanog, and REX-1, the neural stem cell-specific factors, were measured by a real-time PCR (Quantitative real-time PCR Analysis). Specifically, RNA was isolated from the cells induced by dedifferentiation stem cells using a kit (Qiagen RNeasy kit, Qiagen Hilden, Germany), and cNDA was synthesized using 2 ug of RNA, oligo(dT) and reverse transcriptase (Superscript II reverse transcriptase, Gibco). Primer of the OCT-4, Nanog and REX-1 genes shown in Table 3 below and master mix (iQ SYBR Green qPCR Master Mix) were added to each of the synthesized cDNAs and analyzed using a device (Bio-Rad iCycler iQ system, Bio-Rad Laboratories, USA), and the results are shown in FIGS. 4a and 4b, and Table 4.

TABLE 3

| SEQ | | | |
|---|---|---|---|
| ID | | | |
| NO: | Name | Sequence (5→3) | Note |
| 5 | OCT-4_F | TCTCGCCCCCTCCAGGT | |
| 6 | OCT-4_R | CTGCTTCGCCCTCAGGC | |
| 7 | Nanog_F | AAAGAATCTTCACCTATGCC | |
| 8 | Nanog_R | GAAGGAAGAGGAGAGACAGT | |
| 9 | REX-1_F | CAGATCCTAAACAGCTCGCAGAAT | |
| 10 | REX-1_R | GCGTACGCAAATTAAAGTCCAGA | |
| 11 | GAPDH_F | GAGTCCACTGGCGTCTTCAC | |
| 12 | GAPDH_R | TTCACACCCATGACGAACAT | |

TABLE 4

| | | | PLACENTA_ | FIBROBLAST_ |
|---|---|---|---|---|
| | H1 control | HUVEC_iPSC | iPSC | iPSC |
| OCT-4 | 1.0733333 | 1.21 | 1.09 | 1.0923333 |
| Nanog | 1.1233333 | 1.4333333 | 1.39 | 1.1366667 |
| REX-1 | 1.1333333 | 1.4466667 | 1.3666667 | 1.35 |

As can be confirmed in FIG. 4A, the stem cell-specific markers OCT4 and SSEA4 were strongly expressed in the dedifferentiation stem cells. In addition, as can be confirmed in FIG. 4B and Table 4, the content of the intracellular induced pluripotent stem cell specific-protein in the dedifferentiation stem cells was found to be higher than in the human embryonic stem cell line H1 (WiCell, Wisconsin, USA) as a control.

### 3-2. Chromosome Analysis

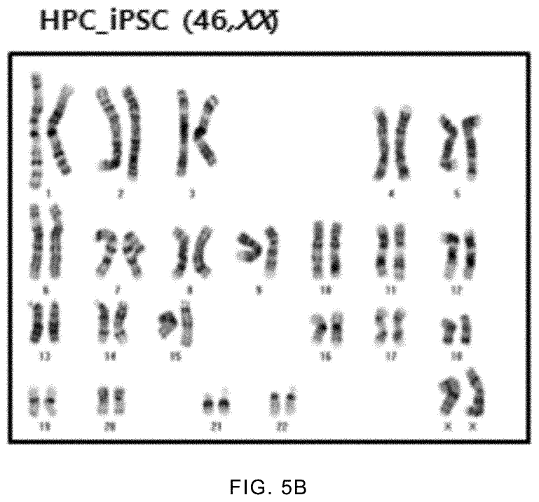

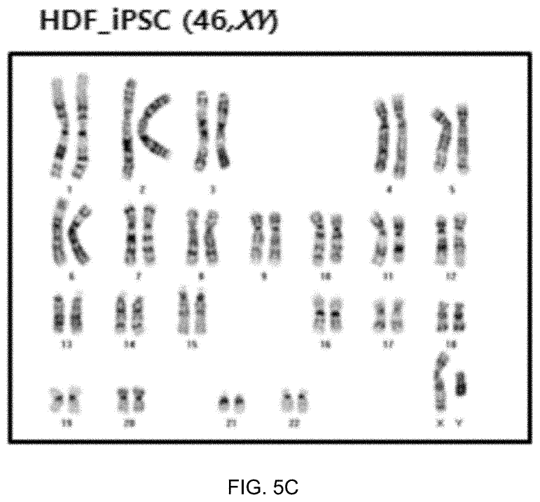

In order to confirm whether the dedifferentiation stem cells maintain normal karyotypes, chromosome analysis was performed to verify the stability. In detail, for the chromosome analysis, 0.1 g/ml of colcemid was treated to the dedifferentiation stem cells at 1.5×10⁶ cells for 3 to 4 hours. Then, 0.25% trypsin-EDTA was treated for 5 minutes to isolate the cells from the culture dish, and then, the cells were incubated in a 0.075M KCl solution at 37° C. for 20 minutes. Then, methanol and acetic acid were mixed at a ratio of 3:1 to fix the cells, and the karyotypes of the established dedifferentiation stem cells were measured at a resolution of 300 band level, and the results are shown in FIGS. 5A to 5C.

As can be shown in FIGS. 5A to 5C, it was confirmed through chromosome analysis that the dedifferentiation stem cells maintained normal karyotypes. The characteristics and stability were verified through the specific markers in the dedifferentiation stem cells established with high efficiency from a total of three various somatic cells using the placenta-derived conditioned medium.

### EXAMPLE 4: CONFIRMATION OF DIFFERENTIATION ABILITY OF DEDIFFERENTIATION STEM CELLS

### 4-1. Confirmation of Differentiation Ability

The differentiation ability was confirmed by forming embryonic cells in vitro to determine whether the dedifferentiated induced pluripotent stem cells have the ability to differentiate into ectoderm, mesoderm, and endoderm. In detail, in order to confirm the differentiation ability in vitro, embryonic cells were formed for 2 weeks in a non-adherent culture vessel, and immunofluorescence was performed to confirm whether the cells could each differentiate into ectoderm, mesoderm and endoderm.

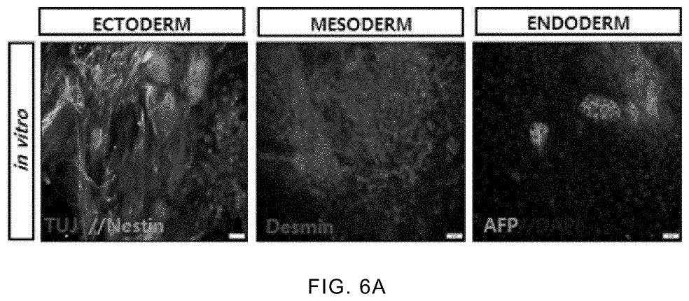

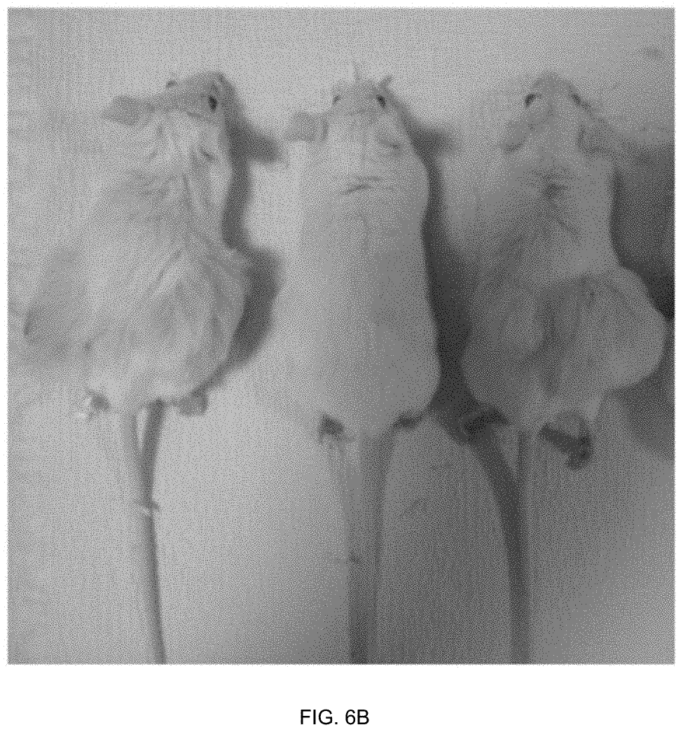

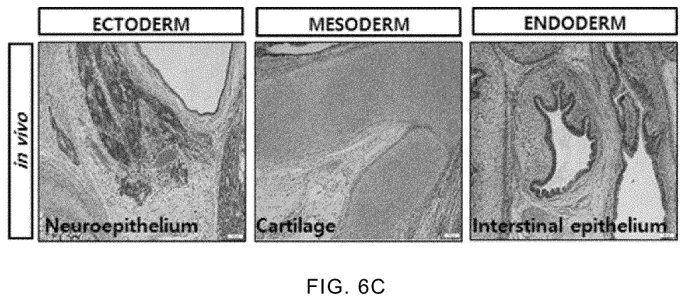

Specifically, after fixing the cells with 4% paraformaldehyde for 10 minutes, 0.1% Triton X100 was infiltrated into the cells for 15 minutes and then blocked with PBS containing 3% horse serum for 1 hour. Then, the primary antibody TUJ1 (COVANCE #MRB-435P), Nestin (Abcam #ab22035), Desmin (Santacruz #sc-14026), and AFP (Santacruz #sc-166335) were diluted at 1:1000 and treated to the cells, and the cells were incubated overnight at 4° C. The next day, the cells were treated with the secondary antibody, incubated at room temperature for 1 hour, to which 4′,6-diamidino-2-phenylindole (DAPI) was added, allowed to stand for 5 minutes in dark conditions, and then observed under a fluorescence microscope. The results are shown in FIG. 6a.

As can be shown in FIG. 6A, the neuroepithelium differentiated into ectoderm was identified using Tuj1 and Nestin markers, and the cartilage differentiated into mesoderm was identified using Desmin as mesodermal marker. In addition, the intestinal epithelium differentiated into endoderm was verified using immunohistochemistry. It was verified by fluorescence immunoassay that differentiation into ectoderm was made from the formation of embryonic cells in vitro, and Desmin as a mesodermal marker and AFP as an endoderm marker were expressed.

### 4-2. Verification of Teratoma Formation

In order to confirm the differentiation ability in vivo, it was verified whether teratomas were formed in immune-deficient mice. In detail, the dedifferentiated induced pluripotent stem cells were injected into the subcutaneous tissue of immune-deficient mice at 1.0×10⁶ cells. After 12 weeks, the formation of teratoma was verified using immunohistochemistry.

In detail, euthanasia was performed using carbon dioxide gas when the formed teratoma was cm³ in size. The teratoma was removed through surgical procedures and fixed in 4% formaldehyde. After dehydration, the tissue was immersed in xylene for a long time to clean the tissue. The tissue was placed in a liquid paraffin container and immersed therein in an oven at 60° C. The tissue was embedded in a mold, attached to a slide glass, dried, then placed in an oven at 60° C. and subjected to deparaffinization for about a day. The eosin (E), in which Harris hematoxylin (H) was exposed at room temperature for 30 seconds, was incubated at room temperature for 1 minute and subjected to histological analysis, and the results are shown in FIGS. 6B and 6C.

As can be confirmed in FIGS. 6B and 6C, it was observed that teratoma was formed in the subcutaneous tissue of the immune-deficient mice. As a result, by confirming the differentiation ability of dedifferentiated induced pluripotent stem cells in vivo and in vitro, the ability of the dedifferentiated stem cells to differentiate into desired cells using the human placenta-derived conditioned medium was verified.

### INDUSTRIAL APPLICABILITY

The present disclosure provides a placenta-derived cell-conditioned medium for inducing dedifferentiation into induced pluripotent stem cells from somatic cells and a method for inducing dedifferentiation using the same.

What is claimed is:

1. A placenta-derived cell-conditioned medium for inducing dedifferentiation into induced pluripotent stem cells (iPS) from somatic cells comprising a human placenta-derived cell culture cultured in a growth medium.

2. The medium of claim 1, wherein the placenta-derived cell is a placenta-derived fibroblast-like cell, which is isolated from the human chorionic plate and cultured.

3. The medium of claim 1, wherein the somatic cell is transformed with a nucleic acid sequence encoding at least one protein selected from the group consisting of OCT4, SOX2, c-Myc and KLF4.

4. The medium of claim 1, wherein the somatic cell is at least one selected from the group consisting of endothelial cells, epithelial cells and placental cells.

5. A method for preparing a placenta-derived cell-conditioned medium for inducing dedifferentiation, comprising the following steps:
a placenta-derived cell culturing step of culturing human placenta-derived cells in a cell growth medium supplemented with a culture solution; and
a culture solution collecting step of collecting the culture solution comprising the human placenta-derived cell culture from the cell growth medium.

6. The method of claim 5, wherein the placenta-derived cell is a placenta-derived fibroblast-like cell, which is isolated from the human chorionic plate and cultured.

7. The method of claim 5, wherein the culture solution is DMEM/F-12.

8. The method of claim 7, wherein the culture solution further comprises a serum replacement agent.

9. A method for inducing dedifferentiation into induced pluripotent stem cells from somatic cells, comprising the following steps:
a somatic cell transformation step of transducing a nucleic acid sequence encoding at least one protein selected from the group consisting of OCT4, SOX2, c-Myc and KLF4 into somatic cells; and
a somatic cell culturing step of culturing the transformed somatic cells in a placenta-derived cell-conditioned medium.

10. The method of claim 9, wherein the placenta-derived cell is a placenta-derived fibroblast-like cell, which is isolated from the human chorionic plate and cultured.

11. The method of claim 9, wherein the somatic cell is at least one selected from the group consisting of endothelial cells, epithelial cells and placental cells.

12. The method of claim 9, wherein the method for inducing dedifferentiation further comprises a stem cell isolation step of isolating stem cells from colonies formed during the somatic cell culturing step.

13. The method of claim 12, wherein the stem cell isolation step is performed by staining with a stem cell label marker.

14. The method of claim 12, wherein the method for inducing dedifferentiation further comprises a stem cell activity verification step of confirming the activity of at least one protein selected from the group consisting of OCT-4, NANOG, SSEA-4 and Tra-81 in the stem cells isolated from colonies.

15. The method of claim 12, wherein the method for inducing dedifferentiation further comprises a stem cell differentiation ability verification step of confirming the differentiation ability into ectoderm, mesoderm or endoderm by forming embryonic cells in vitro using the stem cells isolated from colonies.
